(19) United States
(12) Patent Application Publication
(10) Pub. No.: US 2004/0258774 A1
(43) Pub. Date: Dec. 23, 2004

(54) Method of using a compound of menthol and L-arginine as a preparation for the topical delivery of a 5-phosphodiaesterase inhibitor for the treatment of female sexual dysfunction

(76) Inventors: Ronald J. Thompson, Ft. Thomas, KY (US); James M. Thompson, Cincinnati, OH (US)

Correspondence Address: Donald N. Halgren, 35 Central Street, Manchester, MA, 01944, US

(21) Appl. No.: 10/803,148

(22) Filed: Mar. 17, 2004

Related U.S. Application Data
(63) Continuation-in-part of application No. 10/731,692, filed on Dec. 9, 2003.
Application No. 10/731,692 is a division of application No. 10/004,091, filed on Oct. 23, 2001, now Pat. No. 6,702,733.
Application No. 10/004,091 is a continuation of application No. 09/520,110, filed on Mar. 7, 2000, now Pat. No. 6,322,493.
Application No. 09/520,110 is a continuation-in-part of application No. 09/469,959, filed on Dec. 21, 1999, now abandoned.
Application No. 09/469,959 is a continuation-in-part of application No. 09/414,250, filed on Oct. 7, 1999, now Pat. No. 6,224,541.
Application No. 09/414,250 is a continuation-in-part of application No. 09/340,227, filed on Jul. 1, 1999, now Pat. No. 6,179,775.

(51) Int. Cl. A61K 35/78

(52) U.S. Cl. 424/739; 424/747; 424/742; 424/765; 514/565

(57) ABSTRACT

A manual application of a topical clitoral sensitizing compound combination consisting essentially of L-arginine and a cooling agent comprised of Menthol and a 5-phosphodiaesterase inhibitor, wherein the combination is applicable manually to the clitoris.

This application is a continuation-in-part application of co-pending U.S. patent application Ser. No. 10/731,692 filed 9 Dec. 2003 which is a divisional application of U.S. patent application Ser. No. 10/004,091, filed 23 Oct. 2001, now U.S. Pat. No. 6,702,733 issued 9 Mar. 2004, which is a continuation of application Ser. No. 09/520,110, filed 7 Mar. 2000, now U.S. Pat. No. 6,322,493 which is a continuation-in-part of application Ser. No. 09/469,959 filed on 21 Dec. 1999, which is a continuation-in-part of application Ser. No. 09/414,250, filed on 7 Oct. 1999, now U.S. Pat. No. 6,224,541 which is a continuation-in-part of application Ser. No. 09/340,227, filed on 1 Jul. 1999, now U.S. Pat. No. 6,179,775 each of which are incorporated herein by reference in their entirety.

### FIELD OF THE INVENTION

This invention relates to arrangements for the stimulation of females and more particularly to topical application of specialized stimulatory medicaments.

### BACKGROUND

The compound Sildenafil, known by the trademark Viagra®, is a 5-phosphodiaesterase inhibitor that has proven to be a successful therapy for male erectile dysfunction. Sildenafil is an oral tablet. In a double-blind, placebo controlled clinical study of 583 women with diagnosed female sexual dysfunction; oral sildenafil was no more effective than a placebo. This study was conducted by Dr. Rosemary Basson.

Over several years, compounding pharmacists have attempted to develop a topical preparation of sildenafil to treat women with female sexual dysfunction, but this has proven less than effective because of the size of the Sildenafil molecule. Even when topically applied to the mucous membrane, which is more absorptive than hairy skin, the size of the sildenafil molecule requires the aid of a vehicle, or absorbing agent, to allow absorption of the sildenafil into the corpus cavernosa of the clitoris. The menthol component of the menthol/L-arginine combination acts as an immediate vasodilator of the vessels in the mucous membrane and functions as a lipophilic vehicle to facilitate the transport of the Sildenafil across the mucous membrane.

The L-arginine component of the menthol and L-arginine combination functions to induce the nitric oxide pathway as described in my U.S. Pat. No. 6,702,733, incorporated by reference as stated hereinabove.

L-arginine is a semi-essential amino acid that is a component of a normal diet and stored in cellular tissues in minute amounts. By presenting L-arginine in abundant amounts directly to the corpus cavernosa, dietary intake of L-arginine is unnecessary for normal sexual responsiveness.

### DESCRIPTION OF THE PRESENT INVENTION

The present invention relates to a manually applicable topical preparation comprising a compound of menthol or a menthol substitute, and L-arginine, and any 5-phosphodiaesterase inhibitor as that topical compound. This manually applicable compound is to be manually applied directly to a woman's clitoris, for the treatment of female sexual dysfunction. Such 5-phosphodiaesterase inhibitor in one preferred embodiment may preferably comprise less than 10% of that topical compound.

A first preferred embodiment of a Menthol substitute may be peppermint oil. A second preferred embodiment of a Menthol substitute may be cornmint oil. A third preferred embodiment of a Menthol substitute may be Eucalyptus oil. A fourth preferred embodiment of a Menthol substitute may be Citronella oil. A fifth preferred embodiment of a Menthol substitute may be Camphor oil. A sixth preferred embodiment of a Menthol substitute may be Cinnamon oil.

The invention thus comprises a manual application of a topical clitoral sensitizing combination consisting essentially of L-arginine and a cooling agent comprised of Menthol and a 5-phosphodiaesterase inhibitor, wherein the combination is applicable manually to the clitoris.

The cooling agent may in various preferred embodiments include peppermint oil. The cooling agent may include cornmint oil. The cooling agent may include Eucalypus oil. The cooling agent may include Citronella oil. The cooling agent may include Camphor oil. The cooling agent may include Cinnamon oil. The cooling agent may essentially comprise peppermint oil. The cooling agent may essentially comprise cornmint oil. The cooling agent may essentially comprise Eucalyptus oil. The cooling agent may essentially comprise Citronella oil. The cooling agent may essentially comprise Camphor oil. The cooling agent essentially may comprise Cinnamon oil. The combination may contain less than 10% L-arginine. The combination may contain less than 10% menthol and the combination may contain less than 10% L-arginine and also the combination may contain less than 10% 5-phosphodiaesterase inhibitor.

We claim:

1. A manual application of a topical clitoral sensitizing combination consisting essentially of L-arginine and a cooling agent comprised of Menthol and a 5-phosphodiaesterase inhibitor, wherein said combination is applicable manually to the clitoris.

2. The topical sensitizing combination as recited in claim 1, wherein said cooling agent includes peppermint oil.

3. The topical sensitizing combination as recited in claim 1, wherein said cooling agent includes cornmint oil.

4. The topical sensitizing combination as recited in claim 1, wherein said cooling agent includes Eucalypus oil.

5. The topical sensitizing combination as recited in claim 1, wherein said cooling agent includes Citronella oil.

6. The topical sensitizing combination as recited in claim 1, wherein said cooling agent includes Camphor oil.

7. The topical sensitizing combination as recited in claim 1, wherein said cooling agent includes Cinnamon oil.

8. The topical sensitizing combination as recited in claim 1, wherein said cooling agent essentially comprises peppermint oil.

9. The topical sensitizing combination as recited in claim 1, wherein said cooling agent essentially comprises cornmint oil.

10. The topical sensitizing combination as recited in claim 1, wherein said cooling agent essentially comprises Eucalyptus oil.

11. The topical sensitizing combination as recited in claim 1, wherein said cooling agent essentially comprises Citronella oil.

12. The topical sensitizing combination as recited in claim 1, wherein said cooling agent essentially comprises Camphor oil.

13. The topical sensitizing combination as recited in claim 1, wherein said cooling agent essentially comprises Cinnamon oil.

14. The topical sensitizing combination as recited in claim 1, wherein said combination contains less than 10% L-arginine.

15. The topical sensitizing combination as recited in claim 1, wherein said combination contains less than 10% menthol and said combination contains less than 10% L-arginine and said combination contains less than 10% 5-phosphodiaesterase inhibitor.
